(19) United States
(12) Patent Application Publication
(10) Pub. No.: US 2024/0217939 A1
(43) Pub. Date: Jul. 4, 2024

(54) PROCESSES FOR PRODUCING BIOMONOMERS AND PRECURSORS FOR SAME

(71) Applicant: UOP LLC, Des Plaines, IL (US)

(72) Inventors: Yili Shi, Buffalo Grove, IL (US); Veronica G. Deak, Chicago, IL (US); Kristen E. Allaire, Chicago, IL (US); Joel T. Walenga, Lake Zurich, IL (US)

(21) Appl. No.: 18/506,342

(22) Filed: Nov. 10, 2023

Related U.S. Application Data
(60) Provisional application No. 63/477,859, filed on Dec. 30, 2022.

(51) Int. Cl. C07D 307/68

(52) U.S. Cl. CPC C07D 307/68; B01J 2/06

(57) ABSTRACT

Processes for producing biomonomers and precursors for same. A furoate carboxylation reaction is conducted with particles formed from furoates and an alkali base and optionally a reaction promoter. The particles may be in a slurry. The parties are exposed to gaseous carbon dioxide and heated. The reaction produces dicarboxylates which can be separated and used to produce biomonomers like furan dicarboxylate methyl ester and furan dicarboxylic acid.

### RELATED APPLICATIONS

This application claims priority to U.S. Provisional Patent Application Ser. No. 63/477,859, filed Dec. 30, 2022, the entirety of which is incorporated herein by reference.

### FIELD OF THE INVENTION

This invention relates generally to process for the production of aromatic carboxylic acid compounds including furan dicarboxylic acid and furan dicarboxylate methyl ester, as well as precursors for same, from biomass.

### BACKGROUND OF THE INVENTION

Recently, processes have been developed for producing aromatic carboxylic acids and esters from sugars produced from biomass. These aromatic carboxylic acids and esters can be converted to dicarboxylates which can in turn be utilized to produce monomers like furan dicarboxylate methyl ester (FDME) and furan dicarboxylic acid (FDCA). As is known, these monomers are useful in making polymers and plastics and, since they are at least in part derived from biomass, may be referred to as biomonomers.

These processes are desirable because they provide for the production of the biomonomers as opposed to producing chemicals and monomers from fossil fuel sources. Additionally, the processes are desirable because they may consume carbon dioxide -which is considered a greenhouse gas.

While generally effective for their intended purposes, there is an ongoing desire and need to provide effective and efficient processes for producing biomonomers from biomass derived components and carbon dioxide.

### SUMMARY OF THE INVENTION

The present inventors have found that the particle size and interparticle crystallization of furoate, alkali base, and promoter components have an important role in controlling the activity of the reaction. In particular, smaller particle sizes and intergrown crystals lead to carboxylation reactions that are significantly faster compared with reactions which utilize mixed powders. Thus, forming small furoate feed particles before the carboxylation reactor facilitates the ability to provide smaller reactors and/or achieve higher conversions.

Therefore, the present invention may be characterized, in at least one aspect, as providing a process for producing precursors for bio-monomers by: mixing furoate and an alkali base to form a mixture; forming particles from the mixture, the particles comprising the furoate and the alkali base; and, heating the particles in the presence of carbon dioxide to form dicarboxylates.

The particles may have an average particle size between 20 to 200 microns.

The particles may further include a carboxylate reaction promoter.

The particles may be spherical.

The particles may be formed in a hydrocarbon oil.

The particles may be formed by drying a portion of an effluent from an oxidation reaction zone. The drying may include spray drying and/or an aqueous evaporation.

The mixture may be heated to a temperature between 150° C.to 360° C. at a pressure of up to 6,895 kPa (1,000 psi).

The mixture may be a slurry and the carbon dioxide may be provided as bubbles which flow counter current to the slurry and to the particles.

The process may also include recovering the dicarboxylates and converting the dicarboxylates to furan dicarboxylate dimethyl or furan dicarboxylic acid, or both.

In another aspect, the present invention may be broadly characterized as providing a process for producing furan dicarboxylate methyl ester, or furan dicarboxylic acid from a biomass derived compound by: mixing furoate and an alkali base to form a mixture; forming particles from the mixture, the particles comprising the furoate and the alkali base; heating the particles in the presence of carbon dioxide to form dicarboxylates; recovering the dicarboxylates; and, converting the dicarboxylates to furan dicarboxylate methyl ester, or furan dicarboxylic acid, or both.

The particles may further include a carboxylate reaction promoter.

The particles may be spherical.

The alkali base, a furoate counter ion, or both may be selected from a group consisting of: lithium, sodium, potassium, rubidium, cesium, and mixtures thereof.

The furoate particles may have an average particle size of between 20 to 200 microns.

The mixture is heated to a temperature between 150° C.to 360° C. at a pressure up to 6,895 kPa (1,000 psi).

The mixture may be a slurry. The slurry may be formed in a hydrocarbon having negligible solubility to the furoic acid salts and the alkali base.

Additional aspects, embodiments, and details of the invention, all of which may be combinable in any manner, are set forth in the following detailed description of the invention.

### DETAILED DESCRIPTION OF THE DRAWINGS

One or more exemplary embodiments of the present invention will be described below in conjunction with the following drawing figures, in which:

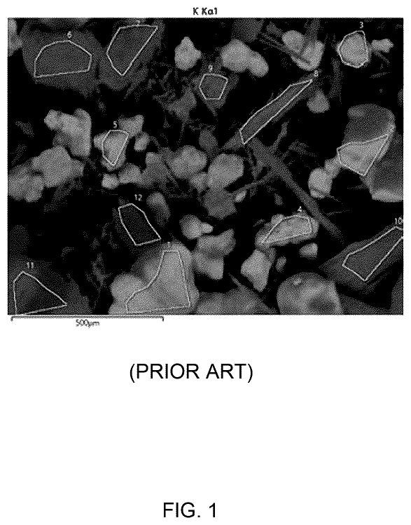

FIG. 1 is a photograph of a conventional mixture of powders used for a carboxylation reaction;

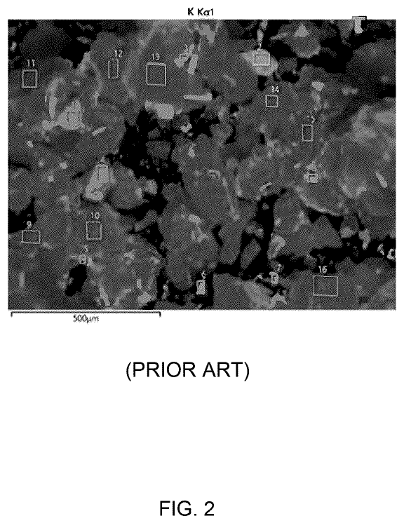

FIG. 2 is a photograph of conventional co-evaporated materials used for a carboxylation reaction; and

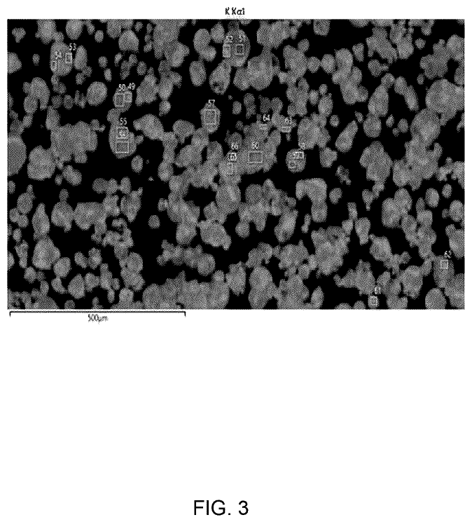

FIG. 3 is a photograph of particles according to the present invention used for a carboxylation reaction.

### DETAILED DESCRIPTION OF THE INVENTION

As mentioned above, the present invention provides processes which use a carboxylation reaction for furoates. Producing furoates from biomass is known. See, U.S. Pat. Nos. 7,572,925 and 8,772,515. As used herein “biomass” includes, but is not limited to, lignin, plant parts, fruits, vegetables, plant processing waste, wood chips, chaff, grain, grasses, corn, corn husks, weeds, aquatic plants, hay, paper, paper products, recycled paper and paper products, and any cellulose, lignin, or combinations thereof containing biological material or material of biological origin. Accordingly, the process is intended to be used as part of an integrated C5 biomass to FDCA/FDME production facility; however, other implementations may be utilized.

In general, the process mixes furoates, such as furoic acid salts, and an alkali base, and optionally any carboxylate reaction promoter to form particles. These particles are heated to a reaction temperature in the presence of a carbon dioxide gas. The furoates are converted to dicarboxylates (FDCA salts) and can then be converted in subsequent chemicals steps into either FDCA free-acid or FDME.

With these general principles in mind, one or more embodiments of the present invention will be described with the understanding that the following description is not intended to be limiting.

Methods according to the present invention include forming a mixture include furoates and an alkali base and then drying the mixture to form particles. The mixture may further include a carboxylate reaction promoter such as a hydrocarbon with an alpha C—H bond, like acetate.

The alkali base may be a metal hydroxide such as lithium hydroxide, sodium hydroxide, potassium hydroxide, rubidium hydroxide, cesium hydroxide, magnesium hydroxide, calcium hydroxide, and mixtures thereof.

A furoate counter ion may include lithium, sodium, potassium, rubidium, cesium, and mixtures thereof.

The alkali base may be at a mole ratio of alkali base to furoate may be from about 1:1 to 2:1, 1:0.1 to 1:1, 1:0.1 to 1:0.5, or 0.1:1 to 1:1.

It is contemplated that the furoates are formed in an oxidation zone in which a biomass derived component is subjected to a selective oxidation reaction process to produce the furoates. The furoates can be separated from the other portions of the effluent mixed with the alkali base, as well as the optional reaction promoter, and then dried to form the particles. The drying may include a spray drying and/or an aqueous evaporation. The particles may or may not be formed in hydrocarbon oil.

Once formed, the particles preferably have an average particle size between 20 to 200 microns, or between 40 to 100 microns, for example, 60 microns. The particles are preferably spherical.

The components of the particles (the furoate and the alkali base) are evenly disbursed or distributed through the individual particles so that each particle is generally homogenous in composition. This is in contrast to processes where two different types of particles are formed at the same time and one type of particle may touch another type of particle. Thus, at least 10 wt %, or at least 20 wt %, or at least 30 wt %, or at least 40 wt %, or at least 50 wt %, or at least 60 wt %, or at least 70 wt %, or at least 80 wt %, or at least 90 wt %, or at least 95 wt %, or at least 99 wt % of the particles are formed by a mixture of at least the furoate and the alkali base.

The particles may be provided in a hydrocarbon oil, for example a hydrocarbon material containing between 5 to 30 carbon atoms per molecule and having paraffinic and/or aromatic functional groups, to form a slurry. In general, the hydrocarbon oil selected for the slurry will have negligible solubility to the furoate and the alkali base. It should be appreciated that the use of a slurry-phase reaction is merely preferred and that other reactors, such as a solid state may be used. If a promoter is not included in the particles, it may be provided with the slurry, or otherwise provided to the particles.

The formed particles are exposed to carbon dioxide gas and heated. If a slurry is used, the carbon dioxide can be provided as bubbles into the slurry. The bubbles may flow counter current to the flow of the slurry and in particular to the flow of the particles.

With the carbon dioxide, the particles are heated to a temperature of between 150 to 360° C., or between 270 to 330° C., at a pressure from about atmospheric up to 6,895 kPa (1,000 psi), or up to 4,826 kPa (700 psig), or up to 4,137 kPa (600 psig) and sufficient heat for a time sufficient to form the dicarboxylates via a carboxylation reaction between the carbon dioxide and the furoate. The reaction time is sufficient to produce the aromatic carboxylic acid compound is from about 1 second to 24 hours, 1 minute to 12 hours, 1 minute to 6 hours, or 1 minute to 1 hour. The process may be continuous, semi-batch or batch reaction process.

The dicarboxylates that are made can include terephthalic acid, naphthalic acid, thiophene dicarboxylic acid, pyridine dicarboxylic acid, carbazole dicarboxylic acid, and dibenzothiophene dicarboxylic acid. In particular, the dicarboxylates may be furan dicarboxylate, and specifically, furan-2,4-dicarboxylatye and/or furan-2,5-dicarboxylatye.

The dicarboxylates may be recovered, for example, by being separated from the slurry. The recovered decarboxylates may be converted to FDME, FDCA, or both. In particular, the produced biomonomers may include one or more of furan-2,5-dicarboxylic acid, furan 2,4 dicarboxylic acid, dimethyl furan-2,5-dicarboxylate, dimethyl furan-2,4-dicarboxylate, and salts thereof. These biomonomers may converted in polymers as is known in the art.

Any separated slurry may be recycled, and heat may be recovered from the separated slurry in a heat exchanger.

The particles, formed from both the furoates and the alkali base, provide an effective and efficient means for producing biomonomers and precursors for same.

### Experiments

Particles according to the present invention (FIG. 3) were used in a carboxylation reaction and compared against a conventional mixture of powders (FIG. 1) and co-evaporated materials (FIG. 2). The feed for the carboxylation reaction was a 1 molar eq. K-furoate, 0.55 molar eq. K₂CO₃, and 0.35 molar eq. KO-Acetate. The feed was heated, in the presence of carbon dioxide, to a temperature of 300° C. for five hours. The conventional mixture of powders (FIG. 1) was found to have a 69% conversion and the co-evaporated materials were found to have a 79% conversion. The particles according to the present invention were found to have a 96% conversion.

### Specific Embodiments

While the following is described in conjunction with specific embodiments, it will be understood that this description is intended to illustrate and not limit the scope of the preceding description and the appended claims.

A first embodiment of the invention is a process for producing precursors for bio-monomers, the process comprising mixing furoate and an alkali base to form a mixture; forming particles from the mixture, the particles comprising the furoate and the alkali base; and, heating the particles in the presence of carbon dioxide to form dicarboxylates. An embodiment of the invention is one, any or all of prior embodiments in this paragraph up through the first embodiment in this paragraph, wherein the particles have an average particle size between 20 to 200 microns. An embodiment of the invention is one, any or all of prior embodiments in this paragraph up through the first embodiment in this paragraph, wherein the particles further comprise a carboxylate reaction promoter. An embodiment of the invention is one, any or all of prior embodiments in this paragraph up through the first embodiment in this paragraph, where the particles are spherical. An embodiment of the invention is one, any or all of prior embodiments in this paragraph up through the first embodiment in this paragraph, wherein the particles are formed in a hydrocarbon oil. An embodiment of the invention is one, any or all of prior embodiments in this paragraph up through the first embodiment in this paragraph, wherein the particles are formed by drying a portion of an effluent from an oxidation reaction zone. An embodiment of the invention is one, any or all of prior embodiments in this paragraph up through the first embodiment in this paragraph, wherein the drying comprises a spray drying. An embodiment of the invention is one, any or all of prior embodiments in this paragraph up through the first embodiment in this paragraph, wherein the drying comprises an aqueous evaporation. An embodiment of the invention is one, any or all of prior embodiments in this paragraph up through the first embodiment in this paragraph, wherein the mixture is heated to a temperature between 150° C. to 360° C. at a pressure of up to 6,895 kPa (1,000 psi). An embodiment of the invention is one, any or all of prior embodiments in this paragraph up through the first embodiment in this paragraph, wherein the mixture comprises a slurry. An embodiment of the invention is one, any or all of prior embodiments in this paragraph up through the first embodiment in this paragraph, wherein the carbon dioxide is provided as bubbles which flow counter current to the slurry and to the particles. An embodiment of the invention is one, any or all of prior embodiments in this paragraph up through the first embodiment in this paragraph, further comprising recovering the dicarboxylates; and, converting the dicarboxylates to furan dicarboxylate dimethyl or furan dicarboxylic acid, or both.

A second embodiment of the invention is a process for producing furan dicarboxylate methyl ester, or furan dicarboxylic acid from a biomass derived compound, the process comprising mixing furoate and an alkali base to form a mixture; forming particles from the mixture, the particles comprising the furoate and the alkali base; heating the particles in the presence of carbon dioxide to form dicarboxylates; recovering the dicarboxylates; and, converting the dicarboxylates to furan dicarboxylate methyl ester, or furan dicarboxylic acid, or both. An embodiment of the invention is one, any or all of prior embodiments in this paragraph up through the second embodiment in this paragraph, wherein the particles further comprise a carboxylate reaction promoter. An embodiment of the invention is one, any or all of prior embodiments in this paragraph up through the second embodiment in this paragraph, where the particles are spherical. An embodiment of the invention is one, any or all of prior embodiments in this paragraph up through the second embodiment in this paragraph, wherein the alkali base, a furoate counter ion, or both are selected from a group consisting of lithium, sodium, potassium, rubidium, cesium, and mixtures thereof. An embodiment of the invention is one, any or all of prior embodiments in this paragraph up through the second embodiment in this paragraph, wherein the furoate particles have an average particle size of between 20 to 200 microns. An embodiment of the invention is one, any or all of prior embodiments in this paragraph up through the second embodiment in this paragraph, wherein the mixture is heated to a temperature between 150° C. to 360° C. at a pressure up to 6,895 kPa (1,000 psi). An embodiment of the invention is one, any or all of prior embodiments in this paragraph up through the second embodiment in this paragraph, wherein the mixture comprises a slurry. An embodiment of the invention is one, any or all of prior embodiments in this paragraph up through the second embodiment in this paragraph, wherein the slurry is formed in a hydrocarbon having negligible solubility to the furoic acid salts and the alkali base.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A process for producing precursors for bio-monomers, the process comprising:
mixing furoate and an alkali base to form a mixture;
forming particles from the mixture, the particles comprising the furoate and the alkali base; and,
heating the particles in the presence of carbon dioxide to form dicarboxylates.

2. The process of claim 1, wherein the particles have an average particle size between 20 to 200 microns.

3. The process of claim 1, wherein the particles further comprise a carboxylate reaction promoter.

4. The process of claim 1, where the particles are spherical.

5. The process of claim 1, wherein the particles are formed in a hydrocarbon oil.

6. The process of claim 1, wherein the particles are formed by drying a portion of an effluent from an oxidation reaction zone.

7. The process of claim 6, wherein the drying comprises a spray drying.

8. The process of claim 6, wherein the drying comprises an aqueous evaporation.

9. The process of claim 1, wherein the mixture is heated to a temperature between 150° C. to 360° C. at a pressure of up to 6,895 kPa (1,000 psi).

10. The process of claim 1, wherein the mixture comprises a slurry.

11. The process of claim 10, wherein the carbon dioxide is provided as bubbles which flow counter current to the slurry and to the particles.

12. The process of claim 1, further comprising:
recovering the dicarboxylates; and,
converting the dicarboxylates to furan dicarboxylate dimethyl or furan dicarboxylic acid, or both.

13. A process for producing furan dicarboxylate methyl ester, or furan dicarboxylic acid from a biomass derived compound, the process comprising:
mixing furoate and an alkali base to form a mixture;
forming particles from the mixture, the particles comprising the furoate and the alkali base;
heating the particles in the presence of carbon dioxide to form dicarboxylates;
recovering the dicarboxylates; and,
converting the dicarboxylates to furan dicarboxylate methyl ester, or furan dicarboxylic acid, or both.

14. The process of claim 13, wherein the particles further comprise a carboxylate reaction promoter.

15. The process of claim 13, where the particles are spherical.

16. The process of claim 13, wherein the alkali base, a furoate counter ion, or both are selected from a group consisting of: lithium, sodium, potassium, rubidium, cesium, and mixtures thereof.

17. The process of claim 13, wherein the particles have an average particle size between 20 to 200 microns.

18. The process of claim 13, wherein the mixture is heated to a temperature between 150° C. to 360° C. at a pressure up to 6,895 kPa (1,000 psi).

19. The process of claim 13, wherein the mixture comprises a slurry.

20. The process of claim 19, wherein the slurry is formed in a hydrocarbon having negligible solubility to the furoate and the alkali base.
